(19) United States
(12) Patent Application Publication
(10) Pub. No.: US 2021/0213137 A1
(43) Pub. Date: Jul. 15, 2021

(54) COMPOSITIONS TO TREAT ULTRAVIOLET (UV)-INDUCED SKIN INJURY

(71) Applicant: Mayo Foundation for Medical Education and Research, Rochester, MN (US)

(72) Inventors: Stephen C. Ekker, Rochester, MN (US); Debabrata Mukhopadhyay, Jacksonville, FL (US); Priyabrata Mukherjee, Edmond, OK (US); Victoria M. Bedell, Philadelphia, PA (US); Luke H. Hoeppner, Austin, MN (US); Stella P. Hartono, Houston, TX (US)

(21) Appl. No.: 17/153,565

(22) Filed: Jan. 20, 2021

Related U.S. Application Data
(62) Division of application No. 16/335,523, filed on Mar. 21, 2019, now abandoned.
(60) Provisional application No. 62/397,734, filed on Sep. 21, 2016.

(51) Int. Cl. A61K 47/68; A61K 47/69; A61P 17/02; A61P 35/00; A61K 9/00; C07K 16/22

(52) U.S. Cl. CPC A61K 47/6845; A61K 47/6929; A61P 17/02; A61K 2039/505; A61K 9/0014; C07K 16/22; A61P 35/00

(57) ABSTRACT

This document relates to materials and methods for administering (e.g., topically administering) one or more vascular endothelial growth factor (VEGF) inhibitors to reduce and/or treat ultraviolet (UV)-induced skin injury. For example, compositions including one or more VEGF inhibitors that can be administered (e.g., topically administered) to a mammal to reduce and/or treat UV-induced skin injury following UV exposure are provided.

### CROSS-REFERENCE TO RELATED APPLICATIONS

This application is a divisional of U.S. application Ser. No. 16/335,523, filed Mar. 21, 2019, which is a National Stage Application under 35 U.S.C. § 371 of International Application Serial No. PCT/US2017/052697, having an International Filing Date of Sep. 21, 2017, which claims the benefit of U.S. Patent Application Ser. No. 62/397,734, filed on Sep. 21, 2016. The disclosures of the prior applications are considered part of (and is incorporated by reference in) the disclosure of this application.

### STATEMENT REGARDING FEDERAL FUNDING

This invention was made with government support under CA148073, CA187035, CA078383, CA136494, DK102232, DK083219, and GM063904 awarded by the National Institutes of Health. The government has certain rights in the invention.

### BACKGROUND

### 1. Technical Field

This document relates to materials and methods for administering (e.g., topically administering) vascular endothelial growth factor (VEGF) inhibitors to reduce and/or treat ultraviolet (UV)-induced skin injury.

### 2. Background Information

Sunlight overexposure (e.g., sunburn) is the greatest risk factor for skin cancer. One-third of Americans report experiencing sunburn within the last year. Childhood sunburns increase the risk of melanoma by at least 60%. Melanoma accounts for more than 70% of skin cancer patient deaths, and only 14% of patients with metastatic disease survive for five years. Unlike many other tumor types, new cases and mortality of melanoma are still rising. In 2016, an estimated 76,380 new cases of invasive melanoma will be diagnosed and mortality is expected to exceed 10,000 for the first time in the US. The estimated annual cost of treating melanoma in the US exceeds $2.3 billion. Sunscreen may offer viable melanoma prevention; however, relying on sunscreen alone is often inadequate.

### SUMMARY

This document provides materials and methods administering (e.g., topically administering) one or more VEGF inhibitors to reduce and/or treat UV-induced skin injury (e.g., sunburn) following exposure to UV light. For example, this document provides materials and methods for topically administering one or more VEGF-inhibitors following UV-exposure to reduce and/or treat acute (e.g., pain, redness, and/or edema) and chronic (e.g., melanoma) symptoms of UV-induced skin injury.

As demonstrated herein, topically administering anti-VEGF antibodies (e.g., Avastin® and/or 2C3) or small molecule VEGF inhibitors (e.g., Nexavar® (sorafenib)) prevented and/or reduced skin injury following UVB exposure. Administration of anti-VEGF antibodies (e.g., Avastin® and 2C3) also prevented and/or reduced melanoma tumor growth.

In general, one aspect of this document features a composition including a vascular endothelial growth factor (VEGF) inhibitor. The composition can be formulated for topical application to the skin of a mammal (e.g., a human). The composition can be a cream. The VEGF inhibitor can be bevacizumab, 2C3, sorafenib, semaxanib, or sunitinib. In some aspects, the VEGF inhibitor is bevacizumab. The bevacizumab can be conjugated to a gold nanoparticle or a silver nanoparticle.

In another aspect, this document features a method for treating a UV-induced skin injury in a mammal. The method includes, or consists essentially of, topically administering a composition including VEGF inhibitor to a mammal exposed to ultraviolet (UV) light, where a symptom of said UV-induced skin injury is reduced. The mammal can be a human. The topical administration can include administering the composition to skin of the mammal. The UV light can include UVB light. The UV-induced skin injury can be sunburn. The symptom of the sunburn can include an acute symptom such as pain, redness, erythema, and/or edema. The VEGF inhibitor can be bevacizumab, 2C3, sorafenib, semaxanib, or sunitinib. For example, the VEGF inhibitor can be bevacizumab. The bevacizumab can be conjugated to a gold nanoparticle or a silver nanoparticle.

In another aspect, this document features a method for treating a UV-induced skin cancer in a mammal. The method includes, or consists essentially of, topically administering a composition including a VEGF inhibitor to a mammal identified as having said UV-induced skin cancer, wherein tumor growth of the UV-induced skin cancer is reduced. The mammal can be a human. The topical administration can include administering the composition to skin of the mammal. The UV-induced skin cancer can be melanoma. The VEGF inhibitor can be bevacizumab, 2C3, sorafenib, semaxanib, or sunitinib. In some aspects, the VEGF inhibitor can be bevacizumab. The bevacizumab can be conjugated to a gold nanoparticle or a silver nanoparticle.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1D show that VEGF is induced early upon exposure to UVB. FIG. 1A. Timeline of the acute and chronic effects of UV-induced skin injury. FIG. 1B. Quantitative PCR was used to measure induction of VEGF transcript in mouse skin upon exposure to various doses of UVB (n=3 mice/group). FIG. 1C. Representative photographs of mouse ears two days after UVB exposure. UVB induces angiogenesis in the ear of mice. FIG. 1D. Systemic administration of VEGF antibody 2C3 reduces edema as shown by 50% decrease in ear thickness (p=0.0101, n=4).

FIGS. 2A-2D show that topical inhibition of VEGF signaling after UVB exposure prevents chronic and acute UVB injury. FIG. 2A. Gold nanoparticles conjugated to VEGF antibody penetrating the skin. FIG. 2B. Topical administration of GNP-VEGF antibody prevents chronic injury on mice skin upon chronic exposure of UVB. 1×1 cm are on the right flank of the mice were exposed to UVB 3 times/week for 3 weeks (n=5). Either vehicle or GNP-VEGF antibody was applied to the area 2 hours after exposure. FIG. 2C. Topical administration of GNP-VEGF antibody is superior compared to topical VEGF antibody or GNP alone in reducing edema. All treatments were applied 2 hours post-UVB exposure. Data representative of 2 experiments, n=5. Ear thickness was measured 2 days after UVB exposure. Avastin® and 2C3 are VEGF antibodies. FIG. 2D. Topical administration of GNP-VEGF antibody or small molecular inhibitor (SMI) of VEGF signaling, sorafenib, reduces edema. All treatments were applied 2 hours post UVB exposure. Data representative of 2 experiments, n=5. Ear thickness was measured 2 days after UVB exposure.

FIGS. 3A-3C show that topical administration of GNP-VEGF Ab limits expansion of primary melanoma after UVB exposure. FIG. 3A. GNP-VEGF antibody applied post UVB exposure prevent growth of primary melanoma tumor. For Tumor+UV group * p<0.05, ** p<0.01 compared to tumor only, § p<0.05 compared to Tumor+UV+Tx group. No significant difference between tumor only and tumor+UV+Tx group at all time points. Data based on 3 experiments (n=5). FIG. 3B. Representative histological images of melanoma tumor stained with CD31 showing that chronic UVB exposure alters vessel density of the primary melanoma tumor and topical treatment with GNP-VEGF antibody attenuated these changes. FIG. 3C. Quantification of vessel density via CD31 staining, a marker of vessels.

FIGS. 4A-4B show a schematic and survival curve for mouse melanoma study.

FIG. 4A. Schematic diagram of experimental setup corresponding to FIG. 3A. All mice were injected with tumor on their right flank on Day 0. Luciferase imaging was done on day 5. Based on luciferase imaging data, mice were assigned to different groups (no UV—tumor only, tumor+UV, and tumor+UV+Tx). Starting on day 6, 1×1 cm area on the tumor was exposed to UVB. Vehicle or treatment was applied 2 hours post UVB exposure. A subset of the mice (n=4) was sacrificed on day 6, 10, 14 and 19 for analysis. FIG. 4B. Survival curve of mice in a separate analogous tumor study to the one represented in FIG. 3A.

FIGS. 5A-5C show that lyophilization of GNP-VEGF Ab preserved their activity. FIG. 5A. GNP-Ab conjugate become progressively less effective with time. GNP-Ab was mixed into the vehicle cream and divided into 4 batches. Each batch was stored in room temperature prior to their use. FIG. 5B & FIG. 5C. Lyophilized GNP-Ab maintained their efficacy in treating UVB radiation injury. Lyophilized Ab was stored at room temperature and resuspended prior to mixing with vehicle cream.

FIG. 6 shows that chronic UV application 3x per week (followed 2 hours later by 2C3:GNP in cream or cream vehicle alone) for 2 weeks.

### DETAILED DESCRIPTION

This document provides methods and materials for reducing and/or treating UV-induced skin injury. For example, this document provides compositions including one or more VEGF inhibitors that can be administered (e.g., topically administered) to a mammal to reduce and/or treat UV-induced skin injury following exposure to UV light. The materials and methods provided herein can be used to reduce the symptoms of UV-induced skin injury. In some cases, one or more VEGF inhibitors can be used to reduce the acute symptoms of UV-induced skin injury. Acute symptoms of UV-induced skin injury (e.g., sunburn) include, for example, pain, redness, erythema, and edema. For example, a composition including one or more VEGF inhibitors can be topically administered to a mammal having a UV-induced skin injury to reduce redness, pain, and edema. In some cases, one or more VEGF inhibitors can be used to reduce the chronic symptoms of UV-induced skin injury. Chronic symptoms of UV-induced skin injury (e.g., sunburn) include, for example, skin cancer (e.g., melanoma), photodamage, and aging (e.g., skin wrinkles). For example, a composition including one or more VEGF inhibitors can be topically administered to a mammal having a UV-induced skin injury to prevent melanoma. The materials and methods provided herein can be used to reduce growth of UV-induced skin cancers (e.g., melanoma, basal cell carcinoma (BCC), and squamous cell carcinoma (SCC)). For example, a composition including one or more VEGF inhibitors can be topically administered to a mammal having melanoma to reduce tumor growth.

When treating a UV-induced skin injury as described herein, the UV-induced skin injury can be any appropriate UV-induced skin injury. Examples of UV-induced skin injury that can be treated with the materials and methods described herein include, without limitation, sunburn, melanoma, non-melanoma skin cancers, melanoma and non-melanoma skin cancer prevention. In some cases, the materials and methods provided herein can be used to treat other skin conditions such as psoriasis, radiation dermatitis, rosacea, and photoaging (e.g., wrinkles).

Any type of mammal having a UV-induced skin injury or at risk of developing a UV-induced skin injury can be treated as described herein. For example, humans and other primates such as monkeys having a UV-induced skin injury can be treated topically with one or more VEGF inhibitors. In some cases, dogs, cats, horses, cows, pigs, sheep, rabbits, mice, and rats can be treated topically with one or more VEGF inhibitors as described herein.

A composition including one or more VEGF inhibitors that can be administered (e.g., topical administered) to a mammal following exposure to any type of UV light. The UV light can be UVA light, UVB light, and/or UVC. The UV light can be natural UV light and/or synthetic UV light. In some cases, exposure to UV light can be confirmed using any appropriate method. For example, a physical examination, phototesting, and/or minimal erythema dose (MED) testing can be used to identify exposure of a human or other mammal to UV light.

Following exposure to UV light, the mammal can be administered or instructed to self-administer one or more VEGF inhibitors (e.g., a composition containing one or more VEGF inhibitors that reduce VEGF polypeptide expression and/or activity). In some cases, a mammal can be identified as having a UV-induced skin cancer (e.g., melanoma). Any appropriate method can be used to identify a mammal as having a UV-induced skin cancer. For example, physical examinations, and/or tissue sample analysis (e.g., fine needle aspirate (FNA); or biopsy procedures such as a shave biopsy, a punch biopsy, excisional biopsy, or an incisional biopsy) can be used to identify a human or other mammal as having a UV-induced skin cancer.

In some cases, a mammal can be administered or instructed to self-administer one or more VEGF inhibitors (e.g., a composition containing one or more VEGF inhibitors that reduce VEGF polypeptide expression and/or activity) prior to being exposed to UV light.

A VEGF inhibitor can be an inhibitor of any appropriate VEGF. Examples of VEGF family members include, without limitation, VEGF-A, placenta growth factor (PGF), VEGF-B, VEGF-C, and VEGF-D. A VEGF inhibitor can be an inhibitor of any isoform of a VEGF. Examples of human VEGF isoforms include, without limitation, VEGF₁₂₁, VEGF₁₂₁b, VEGF₁₄₅, VEGF₁₆₅, VEGF₁₆₅b, VEGF₁₈₉, and VEGF₂₀₆.

A VEGF inhibitor can be an inhibitor of VEGF polypeptide expression or an inhibitor of VEGF polypeptide activity. Example compounds that reduce VEGF polypeptide activity include, without limitation, anti-VEGF antibodies (e.g., Avastin® (bevacizumab) and 2C3), small molecule inhibitors (e.g., Nexavar® (sorafenib)), semaxanib (SU-5416), and sunitinib. In some cases, a VEGF inhibitor can be Avastin®. Examples of compounds that reduce VEGF polypeptide expression include, without limitation, nucleic acid molecules designed to induce RNA interference (e.g., a siRNA molecule or a shRNA molecule), antisense molecules, and miRNAs. Additional VEGF inhibitors can be readily designed based upon the nucleic acid and/or polypeptide sequences of VEGF. Examples of VEGF polypeptides include, without limitation, the human VEGF polypeptide having the amino acid sequence set forth in GenBank® accession Nos: AAA35789 (Version: AAA35789.1; GI: 181971), CAA44447 (Version: CAA44447.1; GI: 37659), AAA36804 (Version: AAA36804.1; GI: 340215), and AAK95847 (Version: AAK95847.1; GI: 15422109). In some cases, a VEGF inhibitor can be an inhibitor of an appropriate tyrosine kinase receptor (e.g., VEGFR (such as VEGFR-1, VEGFR-2, and VEGFR-3), PDGFR, and Raf). Examples of compounds that reduce tyrosine kinase activity include, without limitation, VEGF-A, VEGF-C, VEGF-D, and Sorafenib. A VEGF inhibitor can be a natural inhibitor or a synthetic inhibitor.

A VEGF inhibitor can be conjugated to and/or encapsulated in a delivery vehicle. Examples of delivery vehicles that can be used in the materials and methods provided herein include, without limitation, nanoparticles such as gold nanoparticles (GNPs) and silver nanoparticles (SNPs). In some cases, a VEGF inhibitor (e.g., an anti-VEGF antibody) can be conjugated to a GNP (GNP-Ab). For example, a GNP-Ab can be GNP-Avastin or GNP-2C3.

A VEGF inhibitor can be processed to achieve a desired outcome. In some cases, a VEGF inhibitor can be processed to increase stability. VEGF inhibitor can be stable for extended periods of time (e.g., at least two weeks) at any appropriate temperature (e.g., room temperature). A VEGF inhibitor can be lyophilized to increase stability for long-term storage at room temperature. In some cases, a VEGF inhibitor can be suspended in a sucrose solution. For example, a VEGF inhibitor can be suspended in a sucrose solution and then lyophilized. In cases where a VEGF inhibitor is lyophilized, it can be resuspended (e.g., requiring only the addition of the sterile liquid carrier) prior to use. In some cases, a VEGF inhibitor can be processed to increase dermal penetration. For example, a VEGF inhibitor can have a reduced size to more readily penetrate the skin.

In some cases, one or more VEGF inhibitors (e.g., one, two, three, four, five, or more VEGF inhibitors) can be administered to a mammal to reduce and/or treat a UV-induced skin injury (e.g., sunburn). For example, two or more VEGF inhibitors can be administered to a mammal (e.g., a human) to prevent or minimize a sunburn.

A composition including one or more VEGF inhibitors can be administered to a mammal having a UV-induced skin injury as a combination therapy with one or more additional agents/therapies used to treat a UV-induced skin injury. For example, a combination therapy used to reduce and/or treat a UV-induced skin injury can include administering to the mammal (e.g., a human) a composition including one or more VEGF inhibitors and one or more UV-induced skin injury treatments such as sunscreen (e.g., physical sunscreens and chemical sunscreens), aloe vera, acetaminophen, nonsteroidal anti-inflammatory drugs, steroids, and/or anesthetics. In cases where one or more VEGF inhibitors are used in combination with one or more additional agents used to treat and/or sooth a UV-induced skin injury, the one or more additional agents can be administered at the same time or independently. For example, the composition including one or more VEGF inhibitors can be administered first, and the one or more additional agents administered second, or vice versa.

A composition including one or more VEGF inhibitors described herein can be administered therapeutically (e.g., after exposure to UV light) or prophylactically (e.g., prior to exposure to UV light) to reduce and/or treat a UV-induced skin injury. In some cases, a composition including one or more VEGF inhibitors described herein can be topically administered from about 0 hours (e.g., immediately) to about 48 hours (e.g., from about 2 to about 48 hours, from about 2 to about 36 hours, from about 2 to about 24 hours, from about 2 to about 18 hours, or from about 2 to about 12 hours) after exposure to UV light. In some cases, a composition including one or more VEGF inhibitors described herein can be topically administered no later than about 48 hours (e.g., no later than about 36 hours, no later than about 24 hours, no later than about 18 hours, no later than about 12 hours, no later than about 6 hours, or no later than about 2 hours) after exposure to UV light.

A composition including one or more VEGF inhibitors described herein can be formulated into a topical composition for administration to a mammal having a UV-induced skin injury. A topical composition can be, for example, a cream, foam, gel, lotion, ointment, bandage (e.g., a wet bandage or a dry bandage), wet towelette, or gel matrix. A topical composition including one or more VEGF inhibitors can be applied directly to the skin. In some cases, one or more VEGF inhibitors applied to the skin can penetrate the skin (e.g., transport through the dermis).

A composition including one or more VEGF inhibitors described herein can include one or more pharmaceutically acceptable carriers (additives) and/or diluents. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Effective doses can vary depending on the severity of a UV-induced skin injury, the timing of administration (e.g., before or after UV exposure), the age and general health condition of the subject, excipient usage, the possibility of co-usage with other therapeutic treatments such as use of other agents, and the judgment of the treating physician.

An effective amount of a composition containing one or more VEGF inhibitors can be any amount that reduces the severity of a symptom of a UV-induced skin injury without producing significant toxicity to the mammal. For example, an effective amount of a VEGF inhibitor such as sorafenib can be from about 1 microgram to about 5000 micrograms (e.g., about 1 μg to about 4000 μg, about 2 μg to about 3000 μg, about 3 μg to about 2000 μg, about 4 μg to about 1000 μg, about 5 μg to about 500 μg, about 7 μg to about 250 μg, or about 10 μg to about 100 μg) per cm² of skin. In some cases, an effective amount of sorafenib can be about 12.5 μg per cm² of skin. For example, an effective amount of a VEGF inhibitor such as Semaxenib can be from about 10 micrograms to about 50000 micrograms (e.g., about 12 μg to about 25000 μg, about 15 μg to about 10000 μg, about 20 μg to about 5000 μg, about 25 μg to about 2500 μg, about 50 μg to about 1000 μg, about 75 μg to about 500 μg, or about 100 μg to about 250 μg) per cm² of skin. In some cases, an effective amount of Semaxenib can be about 125 μg to about 137.5 μg per cm² of skin. For example, an effective amount of a VEGF inhibitor such as Avastin® can be from about 2 micrograms to about 5000 micrograms (e.g., about 3 μg to about 4000 μg, about 4 μg to about 3000 μg, about 5 μg to about 2000 μg, about 10 μg to about 1000 μg, about 12 μg to about 500 μg, about 15 μg to about 250 μg, or about 20 μg to about 100 μg) per cm² of skin. In some cases, an effective amount of Avastin® can be about 25 μg per cm² of skin. For example, an effective amount of a VEGF inhibitor conjugated to a delivery vehicle such as GNP-Avastin® can be from about 0.5 micrograms to about 5000 micrograms (e.g., about 1 μg to about 4000 μg, about 2 μg to about 3000 μg, about 3 μg to about 2000 μg, about 4 μg to about 1000 μg, about 5 μg to about 500 μg, about 10 μg to about 250 μg, or about 15 μg to about 100 μg) per cm² of skin. In some cases, an effective amount of GNP-Avastin® can be less than 50 μg per cm² of skin. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, and severity of the UV-induced skin injury may require an increase or decrease in the actual effective amount administered.

The frequency of administration can be any frequency that reduces the severity of a UV-induced skin injury without producing significant toxicity to the mammal. For example, the frequency of administration can be from about once a week to about three times a day, from about twice a month to about six times a day, or from about three times a week to about once a day. The frequency of administration can remain constant or can be variable during the duration of treatment. A course of treatment with a composition containing one or more VEGF inhibitors can include rest periods. For example, a composition containing one or more VEGF inhibitors can be administered daily over a two-week period followed by a two week rest period, and such a regimen can be repeated multiple times. As with the effective amount, various factors can influence the actual frequency of administration used for a particular application. For example, the effective amount, duration of treatment, use of multiple treatment agents, and severity of the UV-induced skin injury may require an increase or decrease in administration frequency.

An effective duration for administering a composition containing one or more VEGF inhibitors can be any duration that reduces the severity of a symptom of the UV-induced skin injury without producing significant toxicity to the mammal. For example, the effective duration can vary from several hours to several days, weeks, or months. In some cases, the effective duration for the treatment of a UV-induced skin injury can range in duration from about one day to about one month. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### Examples

### Example 1: Post-Exposure Anti-VEGF Therapy for UV-Induced Sunburn in Mice

A topical cream was developed to reduce the acute (pain, redness, etc.) and chronic (melanoma) symptoms of UV-induced skin injury (i.e., sunburn). The topical cream-based product promoted penetration of therapeutic VEGF inhibitors through the dermis, thus significantly improving efficacy.

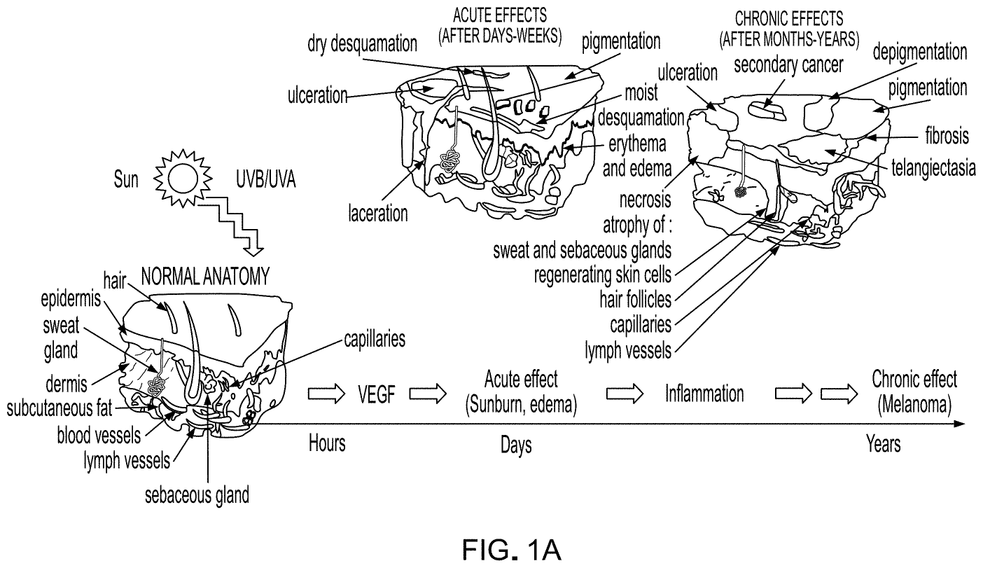

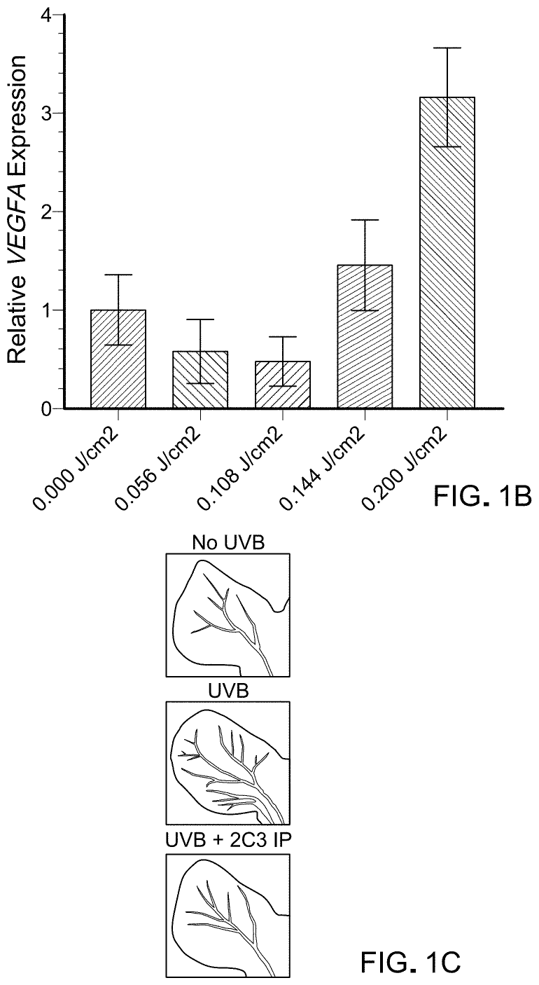

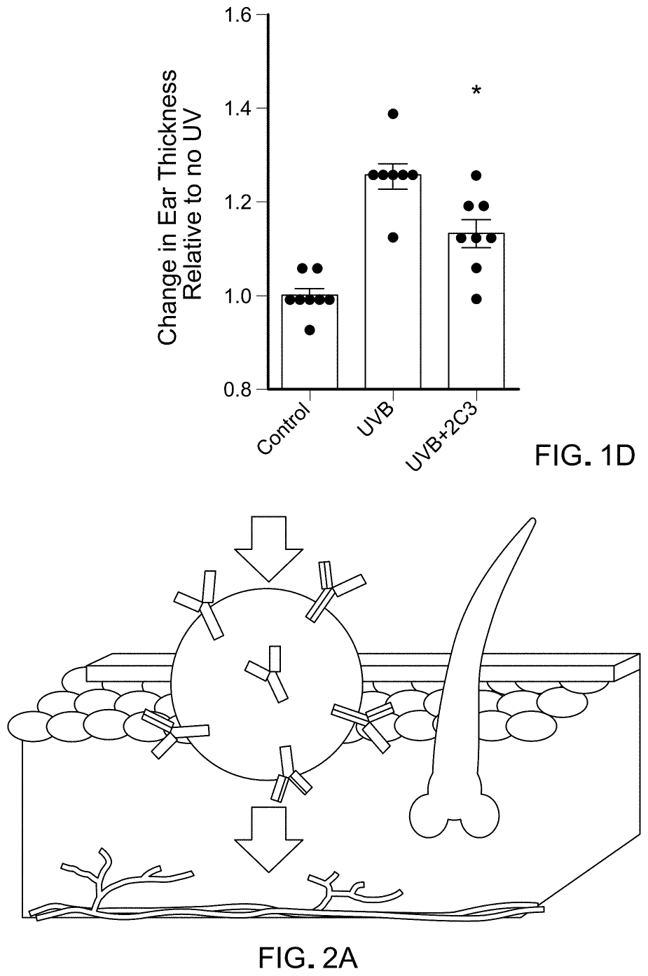

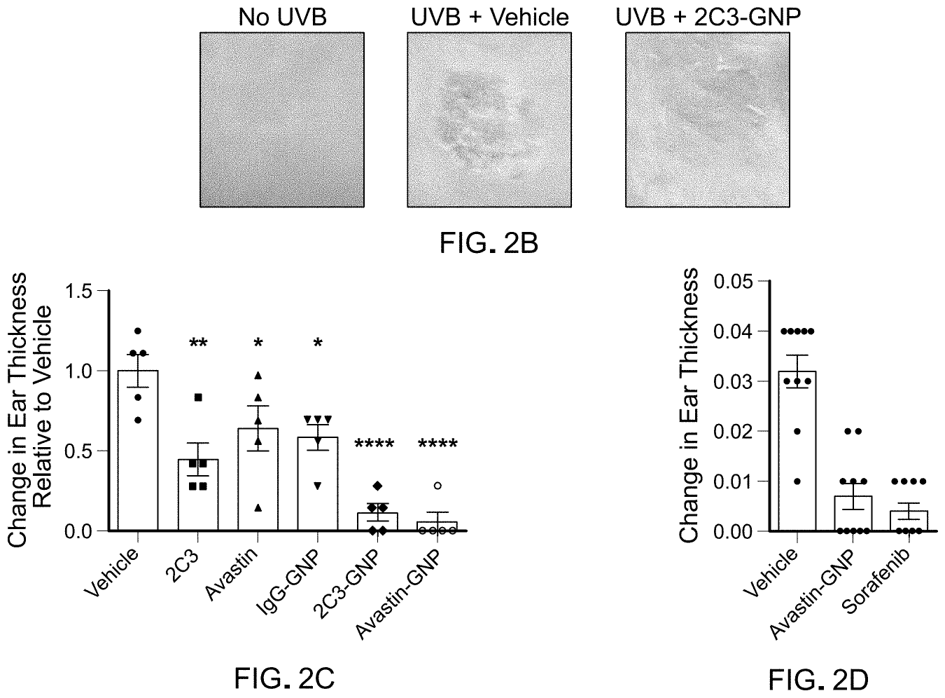

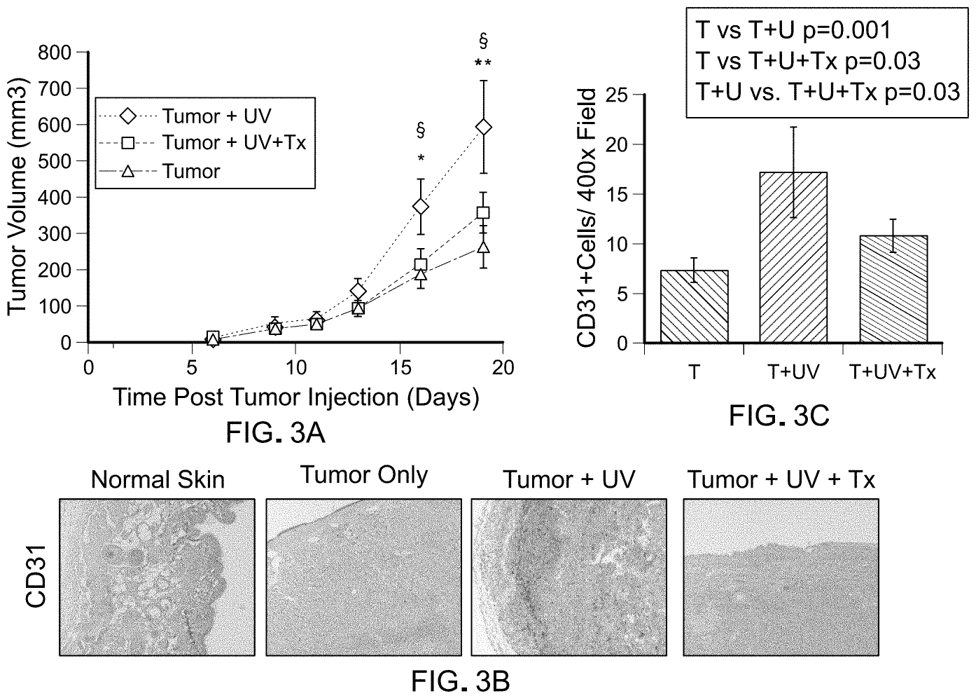

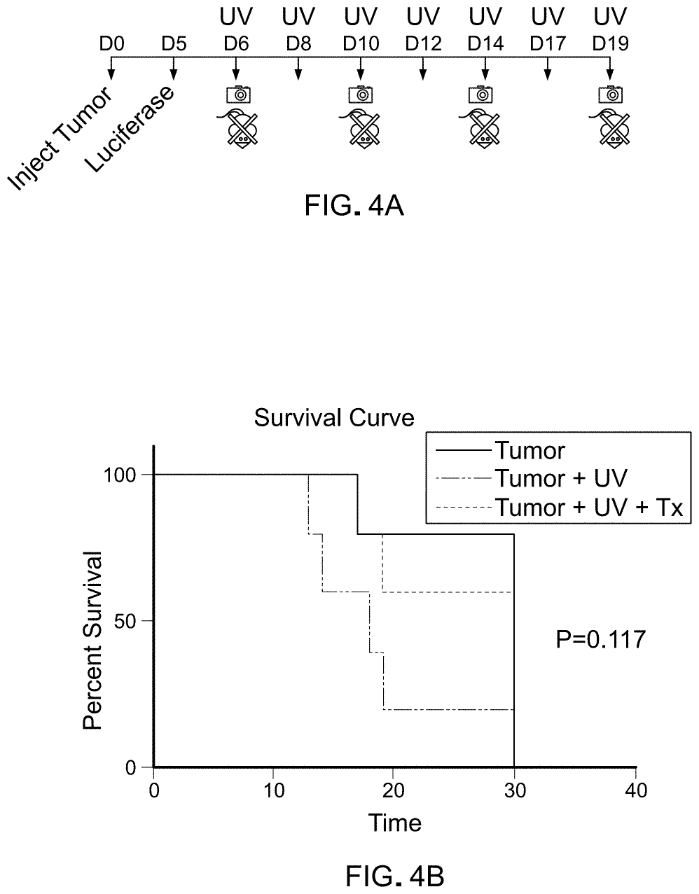

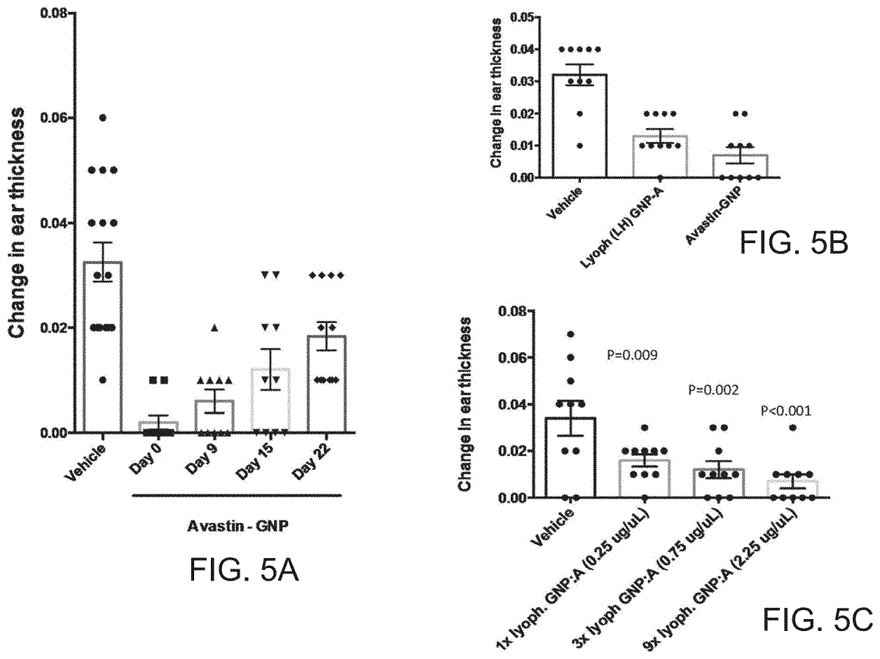

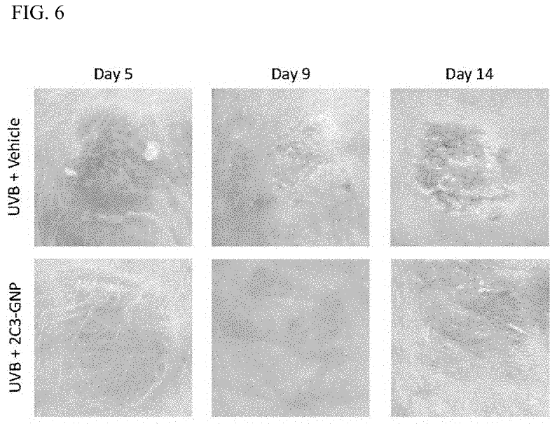

In vivo studies demonstrated that VEGF was induced in the skin in a UV-exposure dependent manner within 48 hours (FIG. 1). Vascular endothelial growth factor (VEGF) was induced by UVB light and works concomitantly with ROS to induce the pathway, and was identified as an upstream regulator of the inflammation pathway. Conjugation of anti-VEGF antibody to gold nanoparticles (aVGNPs) increased its anti-angiogenesis properties compared to VEGF antibody delivered alone (Mukherjee et al., J Nanobiotechnology 5:4 (2007)). Mouse model studies revealed that topical application of aVGNPs after chronic and acute UVB exposure prevented development of erythema and edema that leads to premature aging (FIG. 2A-B). Similarly, topical administration of sorafenib, a small molecule inhibitor of VEGFR-2, inhibited the acute effects of UV-induced skin damage in mice (FIG. 2C). Furthermore, aVGNPs prevented accelerated growth of primary tumor upon chronic UVB exposure in a mouse model of melanoma (FIG. 3).

Taken together, these results demonstrated a clinically relevant method of skin injury prevention following UVB exposure.

### Example 2: Post-Exposure Anti-VEGF Therapy for UV-Induced Sunburn in Humans

Clinical trials are performed in humans. Briefly, 10 patients undergoing minimal erythema dose (MED) evaluation prior to phototherapy are administered aVGNPs as well as various controls in different areas of their UV-exposed skin two hours post-exposure. Skin is evaluated 48 hours later. Photographs of the UV-exposed skin are taken prior to and upon completion of the study. Biopsies are obtained from the aVGNP and vehicle control treated skin.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method for treating a UV-induced skin injury in a mammal, said method comprising:
topically administering a composition comprising vascular endothelial growth factor (VEGF) inhibitor to a mammal exposed to ultraviolet (UV) light;
wherein a symptom of said UV-induced skin injury is reduced.

2. The method of claim 1, wherein said mammal is a human.

3. The method of claim 1, wherein said topical administration comprises administering the composition to skin of said mammal.

4. The method of claim 3, wherein said UV light comprises UVB light.

5. The method of claim 1, wherein said UV-induced skin injury is sunburn.

6. The method of claim 5, wherein said symptom of said sunburn comprises an acute symptom selected from the group consisting of pain, redness, erythema, and edema.

7. The method of claim 1, wherein said VEGF inhibitor is selected from the group consisting of bevacizumab, 2C3, sorafenib, semaxanib, and sunitinib.

8. The method of claim 7, wherein said VEGF inhibitor comprises bevacizumab.

9. The method of claim 8, wherein said bevacizumab is conjugated to a gold nanoparticle (GNP).

10. The method of claim 8, wherein said bevacizumab is conjugated to a silver nanoparticle (SNP).

11. A method for treating a UV-induced skin cancer in a mammal, said method comprising:
topically administering a composition comprising vascular endothelial growth factor (VEGF) inhibitor to a mammal identified as having said UV-induced skin cancer;
wherein tumor growth of said UV-induced skin cancer is reduced.

12. The method of claim 11, wherein said mammal is a human.

13. The method of claim 11, wherein said topical administration comprises administering the composition to skin of said mammal.

14. The method of claim 11, wherein said UV-induced skin cancer comprises melanoma.

15. The method of claim 11, wherein said VEGF inhibitor is selected from the group consisting of bevacizumab, 2C3, sorafenib, semaxanib, and sunitinib.

16. The method of claim 15, wherein said VEGF inhibitor comprises bevacizumab.

17. The method of claim 16, wherein said bevacizumab is conjugated to a gold nanoparticle (GNP).

18. The method of claim 16, wherein said bevacizumab is conjugated to a silver nanoparticle (SNP).
